(19) United States
(12) Patent Application Publication
(10) Pub. No.: US 2022/0061685 A1
(43) Pub. Date: Mar. 3, 2022

(54) METHOD AND SYSTEM FOR DISPLAYING HEART RATE VARIABILITY

(71) Applicant: Covidien LP, Mansfield, MA (US)

(72) Inventor: Paul S. Addison, Edinburgh (GB)

(21) Appl. No.: 17/002,826

(22) Filed: Aug. 26, 2020

(51) Int. Cl. A61B 5/024; A61B 5/1455; A61B 5/00

(52) U.S. Cl. CPC A61B 5/02405; A61B 5/1455; A61B 5/7465; A61B 5/746; A61B 5/742

(57) ABSTRACT

According to an aspect, a method and system for displaying heart rate variability on a display device includes obtaining patient heart rate variability from heart rate information. At least one indicator corresponding to the heart rate variability is superimposed over the heart rate variability on the display device and used to determine a patient status. The patient status is identified as it corresponds to the at least one indicator, such as a color, and communicated to a clinician. The device therefore provides a visual indication of patient status based on the graphic display of the heart rate variability. A minimum threshold value for the heart rate variability is indicated on the display device. An alarm is activated when the minimum threshold value of the heart rate variability is reached and deactivated when the heart rate variability exceeds the minimum threshold value.

### FIELD

The disclosure relates to a method and system for displaying heart rate variability for a neonatal and managing alarms related to low heart rate variability. Pertinent information related to heart rate variability is displayed on a monitor screen in order to provide clear and concise quantitative information to a clinician regarding the status of a patient, typically a neonatal. The displayed information may be used to detect sepsis or the onset of sepsis in neonates.

### BACKGROUND

Medical devices, such as heart rate monitors, can be used to aid a physician or caregiver in patient diagnosis by monitoring a patient condition. Heart rate monitors are used to monitor cardiac signals indicative of expansions, contractions, abnormally slow heart rate, bradycardia, or heart rate variability. Heart rate is the speed of a heartbeat measured by the number of poundings of the heart per unit of time. Heart rate is typically measured in beats per minute and varies according to the body of an individual, absorption of oxygen, and excretion of carbon dioxide. Heart rate variability or heart rate variation may be identified using sensed or paced heartbeats.

Heart rate variability (HRV) is the variation in a time interval between consecutive heartbeats. Typically, the HRV is measured by the variation in the heart beat frequency (i.e., the heart rate). Alternatively, heart rate variability may be measured as a temporal variation in beat-to-beat interval. HRV is a measurable quantity used to assess the health of a patient or individual. HRV may include information about changes in intra-beat cardiac activity, changes in heart rate activity, and may be used to identify patients at an elevated risk of arrhythmia and to identify patients at an increased probability of death.

In a healthy individual, HRV should increase during relaxing activities and decrease during stress. Thus, HRV tends to be higher when the heart is beating slowly and lower when the heart is beating quickly (e.g., exercise, stress). While the HRV level may naturally fluctuate from day to day based on activity level and/or stress level, when an individual is in a disease state or under stress, low HRV may persist.

HRV metrics have shown good correlation with a disease state or stress of a patient. By monitoring HRV, patients at an elevated risk of cardiac arrhythmia or death may be identified. For example, HRV has been used to detect sepsis and the onset of sepsis in neonates, as well as other conditions, such as encephalopathy and the identification of pain. Information about HRV may also be used to determine patients needing cardiac therapy. Based on information obtained from monitoring HRV, at-risk patients may be identified.

Therefore, a need exists for incorporating an HRV metric into monitors in order to aid in observing the health status of a patient.

### SUMMARY

According to one embodiment, a method of displaying heart rate variability includes obtaining a heart rate including at least patient heartbeats; determining a heart rate variability level using the heart rate; displaying the heart rate on a display device; and displaying the heart rate variability on the display device. The method further includes superimposing at least one indicator corresponding to the heart rate variability over at least the heart rate variability on the display device; and identifying a patient status. The patient status corresponds to the at least one indicator superimposed over at least the heart rate variability.

The method for displaying heart rate variability and managing heart rate variability alarms includes obtaining a heart rate value from a patient using information about patient heart beats over a time period; obtaining a heart rate variability using a standard deviation of a heart rate variability from a mean value over the time period; and displaying the heart rate value and the heart rate variability on a display device. The method further identifies a minimum threshold value for the heart rate variability and marks the minimum threshold value on the display device. Then, at least one indicator corresponding to the minimum threshold value is superimposed over the heart rate variability on the display device. The method further includes the steps of quantifying a patient status to a clinician corresponding to the at least one indicator superimposed over the heart rate variability, and triggering an alarm into an on position when the heart rate variability is equivalent to or below the minimum threshold value and an off position when the heart rate variability exceeds the minimum threshold value.

The system for displaying heart rate and heart rate variability includes a heart rate monitor configured to receive information indicative of at least a heart rate of a patient; a processor circuit coupled to the heart rate monitor; and a display device. The processor circuit is configured to receive a first signal with a heart rate of a patient, receive a second signal having a heart rate variability of the patient, wherein the heart rate variability is a standard deviation of the heart rate variability from a mean value over a period of time; and then display the heart rate and the heart rate variability on the display device. The display device includes at least one indicator corresponding to the heart rate variability superimposed on the display device such that a patient status corresponding to the at least one indicator is identified when the second signal is above or below a threshold value.

These and other features of the systems and methods of the subject disclosure will become more readily apparent to those skilled in the art from the following detailed description of the preferred embodiments taken in conjunction with the drawings.

### BRIEF DESCRIPTION OF DRAWINGS

The following descriptions should not be considered limiting in any way. With reference to the accompanying drawings, like elements are numbered alike:

FIG. 1 depicts a flowchart of a general method according to an exemplary embodiment;

FIG. 2 depicts a flowchart of a general method according to an exemplary embodiment;

FIG. 3 is a diagram showing a system for measuring heart rate variability of a user;

FIG. 4 depicts a graph illustrating a heart rate variability around a mean value in accordance with an exemplary embodiment;

FIG. 5 depicts a graph illustrating the heart rate variability of FIG. 4 and marked with a double ended arrow in accordance with an exemplary embodiment;

FIG. 6 depicts a graph illustrating the heart rate variability of FIGS. 4 and 5 with only one of the HRV indicative lines marked in accordance with an exemplary embodiment;

FIG. 7 depicts a graph illustrating the heart rate plot and the heart rate variability plot of FIGS. 4-6 separated for viewing purposes in accordance with an exemplary embodiment;

FIG. 8 depicts a graph with the heart rate plot of FIG. 7 and an alternative display arrangement of the heart rate variability plot, displayed here as a magnitude from zero in accordance with an exemplary embodiment;

FIG. 9 depicts the heart rate plot and the heart rate variability plot of FIG. 8 incorporating a minimum threshold value as part of the region in the heart rate variability plot, in accordance with an exemplary embodiment;

FIG. 10 depicts the heart rate plot and the heart rate variability plot of FIG. 9 with the threshold area value being marked for triggering an alarm in accordance with an exemplary embodiment;

FIG. 11 depicts the heart rate on a first plot, the heart rate variability on a second plot wherein an indicator may be superimposed on the plot to identify a patient status, and a bar chart display in accordance with an exemplary embodiment;

FIG. 12 depicts the heart rate plot, heart rate variability plot, and bar chart of FIG. 11 wherein the superimposed region between the indicative heart rate variability lines is marked in accordance with an exemplary embodiment; and

FIG. 13 depicts various levels of heart rate variability having different indicator values corresponding to patient risk levels in accordance with an exemplary embodiment of the disclosure.

### DETAILED DESCRIPTION

A detailed description of one or more embodiments of the disclosed method and system is presented herein by way of exemplification and not limitation with reference to the Figures.

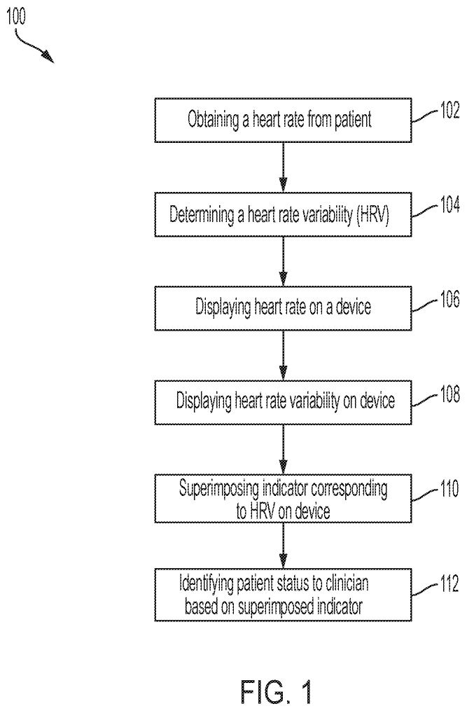

The present disclosure relates to a method 100 and system 200 for displaying heart rate and heart rate variability and for managing heart rate variability alarms in order to monitor and track a patient's health status or stress levels. Pertinent information related to heart rate variability is displayed on a monitor screen in order to provide clear and concise quantitative information to a clinician regarding the status of a patient, typically a neonatal. Referring to FIG. 1, a flowchart depicts a method 100 according to an exemplary embodiment for displaying heart rate variability. The method 100 includes obtaining a heart rate 102 from a patient. From the heart rate, a heart rate variability (HRV) 104 may be determined. Both the heart rate and HRV are displayed on a device. The heart rate is displayed 106 and then the HRV is displayed 108 on the device. At least one indicator corresponding to an HRV level is superimposed 110 on the device. Using the superimposed indicator over the HRV, it is possible to identify a patient status 112.

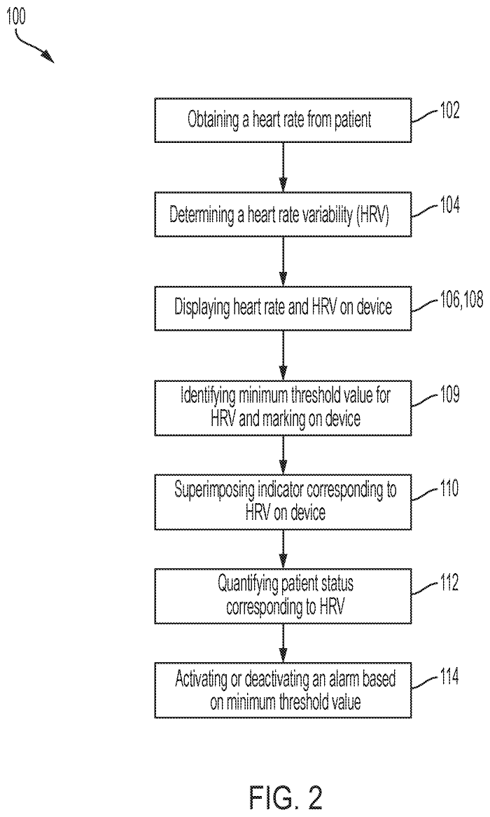

As will be described below, various plots and graphing features may be employed to display both the heart rate and HRV on a device 106, 108. As seen in FIG. 2, the method further includes identifying a minimum threshold value 109 for HRV and marking the same on the device. By incorporating the data sets of heart rate and HRV, a clinician is able to determine a patient status 112. An indicator corresponding to the HRV is superimposed 110, and the patient status corresponding to the HRV may be quantified 112. Based on the minimum threshold value denoted on the graph of the HRV, an alarm could be activated or deactivated 114 when the HRV is below or above, respectively, the minimum threshold value.

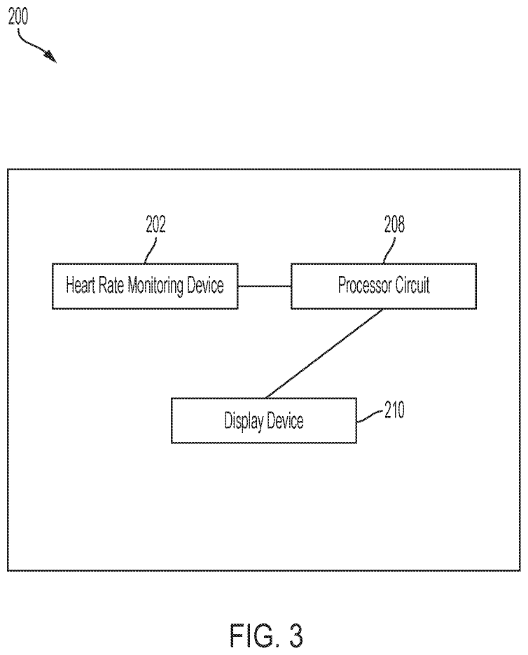

Turning to FIG. 3, the system 200 for displaying heart rate and heart rate variability includes a heart rate monitoring device 202, such as a pulse oximeter monitor, for example, that is configured to receive information indicative of at least a heart rate of a patient and used to measure the heart rate 204. The heart rate includes at least a plurality of patient heartbeats or information about patient heart beats over a time period. The heart rate monitoring device 202 may include at least one sensor that is applied to the patient and collects heart rate data. The data and information indicative of at least the heart rate of the patient is received and transmitted to a processor circuit 208 coupled to the heart rate monitoring device 202. The processor circuit 208 receives a signal related to a patient heart rate, and a signal comprising a heart rate variability for the patient based on the patient heart rate, the heart rate variability being a standard deviation of the heart rate variability from a mean value over a period of time. Once the processor circuit 208 receives the data, the data is transmitted for display on the display device 210. The display device 210 such as a monitor screen displays at least the heart rate 204 such that it may be viewed by a clinician.

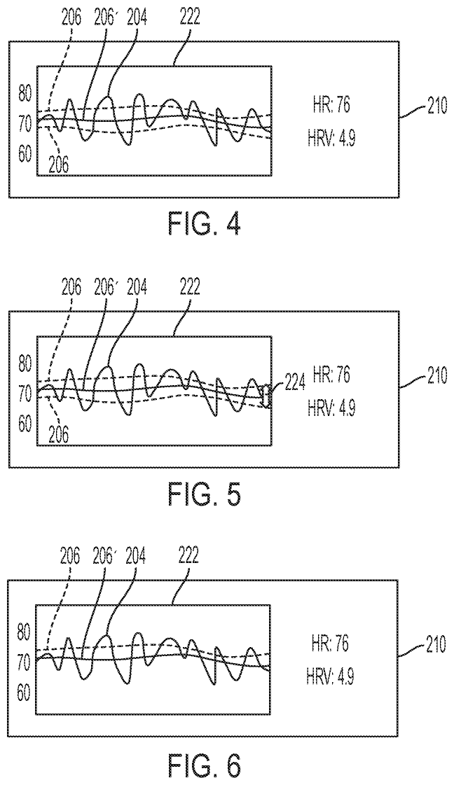

Once the heart rate 204 is obtained, an HRV level 206 may be determined from this collection of data points. Both values, the heart rate 204 and HRV 206 are displayed on a graph 222 disposed on a display device 210 such as a monitor screen, for example. The pulse oximeter is able to measure heart rate 204 over any time period and then plot the results over time, as seen in FIGS. 4-7, for example. The HRV 206 of a subject displayed on the display device 210 may be the standard deviation of the heart rate variation from a mean value 206′ over a time period T. From this additional collection of data, the HRV 206 may also be plotted over time and may be displayed around the mean value as two dotted lines, seen in FIGS. 4 and 5. For example, as seen in FIGS. 4-7, the standard deviation 206 is represented on the same plot 222 as the mean value 206′ and the heart rate 204. Both the value of the heart rate 204 and the value of the HRV 206 may be calculated in the same units, e.g., beats per minute or bpm. The HRV 206 is obtained using a mean value of the heart rate 204 value over the time period T.

The heart rate 204 may be displayed as a function of beats per minute over a time period. From the heart rate 204, the HRV 206 is displayed. A first collection of data is positioned on a first chart as a first line, using a dotted line, for example. A second collection of data, also positioned on the first chart, is a second dotted line below the first line. This represents the HRV 206 around the mean value 206′. As the window is translated over the signal (e.g., in steps of 1 second, 10 seconds, 30 seconds, 1 minute, etc.), a moving HRV metric is determined. The heart rate 204 and the HRV 206 are displayed on at least a first graph 222 of the display device 210 such that both values 204, 206 are visible on the same area of the display device 210.

The HRV 206 may also be displayed at the current time on the signal display and marked. For example, as shown in FIG. 5, a display option for the HRV 206 at the current time may be employed by marking this value with a signal, such as a double ended arrow 224 on the graph 222. This quickly and easily identifies to the clinician the most up to date measured HRV value 206. Alternatively, in another display option shown in FIG. 6, the graph 222 would show only one of the two indicative lines 206 on the plot from FIGS. 4 and 5. Displaying one of the indicative lines 206 may ease viewing capabilities for a clinician.

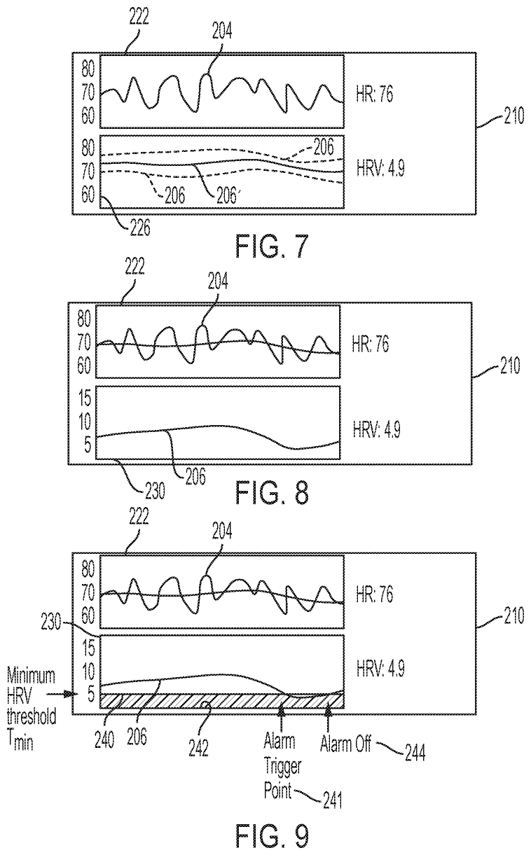

In yet another display option seen in FIG. 7, the plot of the heart rate 204 and the plot of the HRV 206 may be separated for viewing purposes. In that case, the heart rate 204 is displayed on a first graph 222 of the display device 210 and the HRV is displayed on a second graph 226 of the display device 210 separate from the first graph. With this display option, the heart rate 204 and the HRV 206 are separately graphed and plotted on the display device 210.

Also, if different units of measurement are used for heart rate 204 than for HRV 206, it may be desirable to separate the plots. For example, the differences could relate to a normalized ratio such as high frequency power and low frequency power; a percentage of beat intervals over a certain period; an entropy measure; a peak frequency; a correlation dimension; etc. HRV may be measured by one of beats per minute, frequency power, percentage of beat intervals over a period of time, entropy measure, peak frequency, and correlation dimension, for example. Referring to FIG. 6, the HRV 206 is plotted with the heart rate 204 still shown in the HRV 206 plot. It may be possible or desirable for the clinician to remove the heart rate 204 data from this plot, as seen in FIG. 7, where the heart rate 204 plot is separated from the HRV 206 plot that results in a different viewing option for the clinician than that of FIGS. 4-6, for example.

In an alternative arrangement for displaying heart rate 204 and HRV 206, FIG. 8 shows a display device 210 where the heart rate 204 and HRV 206′ are disposed on a first graph 222 and then the HRV 206 is displayed on a second graph 230 adjacent the first graph 222, yet different from the FIG. 7 display, where HRV 206 is displayed as a magnitude from zero. This display option allows for marking of indicative thresholds as will be further described below. As also seen in FIG. 8, the heart rate 204 data is not displayed on the second graph 230, but may still be referred to even as it corresponds to the HRV 206 in the first graph 222 on the display device 210.

As will be described below with respect to FIGS. 9-13, the method 100 and system 200 further include the step of superimposing at least one indicator corresponding to the HRV on the display device 210. A minimum threshold value 240 for HRV levels is identified and annotated on at least one graph 230 of the display device 210. By superimposing at least one indicator 242 related to a minimum threshold value 240 corresponding to a critical HRV level over the HRV 206 plotted on the display device 210, it is possible to identify a patient status to a clinician based on how close the HRV 206 reaches the minimum threshold value 240. The patient status is quantified to a clinician using this metric.

Particularly, the patient status corresponds to the level of the at least one indicator 242 superimposed over the HRV value 206. If the HRV value 206 reaches the minimum threshold level 240, an alarm is triggered, for example, in order to notify the clinician that the patient status is not in the range of a healthy level. The trigger point 241 for an alarm is based on the minimum threshold value 240. When this occurs, the alarm is activated into an on position 241 while the HRV 206 is equivalent to or below the minimum threshold level 240. The alarm is activated into an off position 244 when the HRV 206 exceeds the minimum threshold level 240, as seen in FIG. 9.

Generally, the at least one indicator superimposed on the display device 210 and the HRV 206 may include at least one color identified, or at least one line disposed, on at least one region of the display device 210 configured to indicate that the level of the HRV 206 has decreased below a minimum threshold value 240 over a period of time. As seen in the plot of FIG. 9, a minimum HRV threshold 240 is identified and denoted with a line on the graph. As such, by denoting a minimum HRV threshold 240 and displaying the HRV 206 adjacent the minimum HRV threshold 240, the minimum HRV threshold 240 is compared with the HRV 206 and the patient status may be determined by a clinician. This provides clear and concise quantitative information to the clinician regarding the status of the patient.

As seen in FIG. 9, the entire area 242 disposed below the minimum HRV threshold value 240 may be shaded a color or shade that is different from the rest of the graph 230 that denotes a healthier HRV level. As such, when the HRV 206, displayed here as a magnitude from zero, dips below the minimum HRV threshold 240 level and into the colored display section 242 of the graph 230, an alarm would be triggered 241 as an indication to the clinician that the HRV 206 is measuring in an area that should be of concern. With this, at least part of the HRV 206 is less than or displayed below the minimum HRV threshold 240. The step of triggering an alarm indicates that the patient is at an increased risk of mortality or morbidity when the at least one color on the display device, for example, is superimposed on the signal and the HRV 206 is maintained below the threshold value 240. When the HRV 206 measures below the minimum HRV threshold 240, an alarm would be triggered. When the HRV 206 increases and measures to a level above the minimum HRV threshold 240, the alarm is deactivated and turns off 244. Turning off 244 or deactivating the alarm requires that at least part of the HRV 206 is at a level higher than, and displayed above, the minimum HRV threshold 240.

By marking the minimum HRV threshold 240 on the graph 230, indicative levels associated with poor patient outcomes, such as morbidity or mortality may be monitored. Herein, an HRV 206 associated with poor patient outcomes or a key morbidity may be marked on the plot in order to quantify a patient status to a clinician. Sounding the alarm would indicate to the clinician monitoring the patient that the health status of the patient is reaching a critical level. Alternatively, an upper or maximum threshold value may also be set in order to indicate excessive HRV.

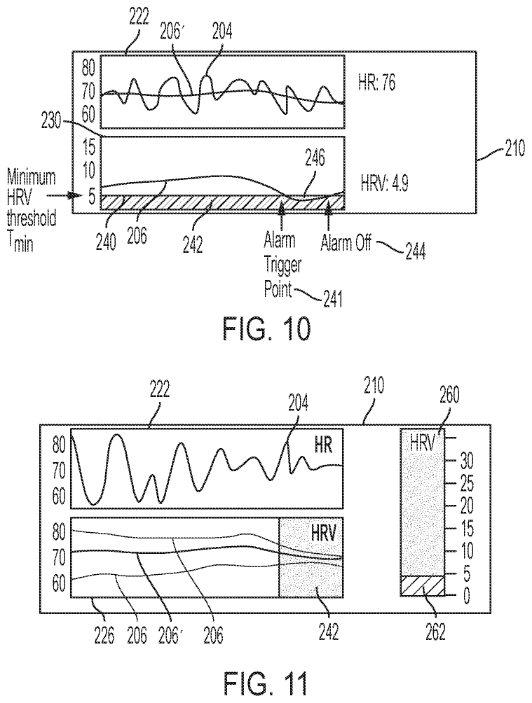

FIG. 10 is similar to that of FIG. 9, but further establishes that it may be possible to integrate the area 246 under the threshold curve (from FIG. 9) and only activate the alarm once the area 246 is above a preset value. For example, if the HRV 206 repeatedly measures less than the minimum threshold value 240 such that the alarm is triggered 241, and then quickly rises such that the HRV 206 measures more than the threshold value 240, the alarm turns off 244. Monitoring this fluctuation over time would avoid short term nuisance alarms that are triggered each time the HRV 206 dips below the threshold minimum value, particularly for brief periods. As such, the continued observation of the HRV value and notification of the area under the threshold 246 continually reached would activate the alarm rather than the area 242 that automatically triggers the alarm 241.

Additional displays for heart rate and HRV are seen in FIGS. 11-13. The HRV 206 in FIG. 11 in addition to being displayed on graph 226, may be transposed as a magnitude of zero and displayed on a bar chart or bar graph 260. By way of example, bar chart 260 is positioned on the display device 210 adjacent the heart rate 204 measurement on graph 222 and the HRV 206 measurement on graph 226. This display option provides multiple graphs for viewing the heart rate 204 and HRV 206 for comparison and easy viewing purposes. As with the above description, FIG. 11 also incorporates a display device 210 with at least one indicator superimposed on a colored background when the HRV 206 decreases over a period of time below a threshold value, for example. In this display option, the minimum threshold value indicating a critical HRV 206 may be marked by a colored area 242 that is noticeably different from the healthier HRV levels. As with the above description, the system 200 is configured to trigger or activate an alarm when the HRV 206 decreases below the threshold value into the critical area 242 and deactivate the alarm when the HRV 206 increases and does not reach the critical area 242. The values may be displayed on a graph as well as on a bar chart 260, as shown in FIG. 11.

As shown in FIGS. 11-13, the display 210 may superimpose colors on the signal indicating that the HRV 206 level is below a threshold 242 which corresponds to a state where the patient may be at an increased rate of mortality or morbidity. This level is indicated in FIGS. 11-13 as being a different color than the other levels, for example. As shown in FIG. 11, the HRV 206 is decreasing over time and thus it is necessary to indicate to a clinician the patient status. This may be done by superimposing 242 the signal on a background having a different color, such as red, than the rest of the display area. As such, once the HRV 206 dips below the threshold value, the signal is displayed on the red background, for example. This aides in quantifying or identifying the increased risk of mortality or morbidity in a patient.

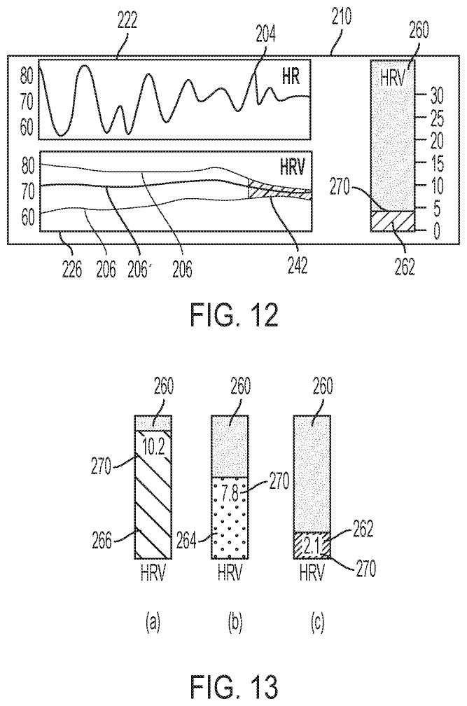

FIG. 12 represents a scenario where only the region or area 242 between the indicative HRV lines 206 of concern are shaded a different color than the rest of the HRV values. As with FIG. 11, FIG. 12 also includes a bar chart 260 representing the same HRV value 206 as indicated on the graph 226. As with the graph 226, the bar chart 260 also has a different color, such as red, for indicating the threshold HRV level 262 that is reached such that when the HRV 270 is represented on the bar chart 260, if the HRV 270 is in the threshold area 262, an alarm may be triggered. This may be an adjunct or alternative display which makes the HRV level 270 or status more noticeable to the clinician viewing the screen 210 in order to identify a patient status corresponding to the HRV level.

FIG. 13 represents three different levels of risk 262, 264, 266 corresponding to HRV 270 on the bar chart 260. As can be seen in FIG. 13, each bar chart 260 includes a different color marked depending on the different HRV 270 level of risk and thus corresponding to a different patient status. The plurality of colors for the bar chart 260 includes a first bar color 266 configured to display or indicate a first level of HRV risk, a second bar color 264 configured to display or indicate a second level of HRV risk, the second level 264 being lower than the first level 266, and a third bar color 262 configured to display or indicate a third level of HRV risk, the third level 262 being lower than both the first and second levels 264, 266 of HRV risk, wherein each of the first bar color 266, the second bar color 264, and the third bar color 262 represent different colors to aid in clinician visibility of the HRV and rapid identification of a patient status quantified accordingly.

By way of example, a high (healthy) HRV status may be colored a first bar color 266 such as green. A medium HRV status, or a level between a clinically high HRV level and a clinically low HRV level, may be colored orange, for example, or a second color 264 separate and distinguishable from the first color 266. A low HRV status, or a level below the medium HRV status may be designated with a third color 262, such as red, separate from the first and second colors 266, 264 in order for a clinician to easily and readily identify a patient status. In accordance with the method, an alarm may be activated when the HRV representing one of the colors, e.g., red, is represented on the bar chart 260 at a decreased level, and the alarm is deactivated when the HRV representing another of the colors, e.g., green is represented on the bar chart 260 at an increased level.

The term “about” is intended to include the degree of error associated with measurement of the particular quantity based upon the equipment available at the time of filing the application.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

What is claimed is:

1. A method of displaying heart rate variability, the method comprising the following steps:
obtaining a heart rate including at least patient heartbeats;
determining a heart rate variability level using the heart rate;
displaying the heart rate on a display device;
displaying the heart rate variability on the display device;
superimposing at least one indicator corresponding to the heart rate variability over at least the heart rate variability on the display device; and
identifying a patient status, wherein the patient status corresponds to the at least one indicator superimposed over at least the heart rate variability.

2. The method of displaying heart rate variability of claim 1, wherein the at least one indicator includes at least one color or at least one line disposed on at least one region of the display device.

3. The method of displaying heart rate variability of claim 1, wherein the step of obtaining a heart rate is conducted using a pulse oximeter monitor.

4. The method of displaying heart rate variability of claim 1, further including the step of denoting a minimum heart rate variability threshold and the heart rate variability indication is displayed adjacent the minimum heart rate variability threshold; and comparing the minimum heart rate variability threshold with the heart rate variability.

5. The method of displaying heart rate variability of claim 4, further including the step of triggering an alarm if at least part of the heart rate variability is displayed below or is less than the minimum heart rate variability threshold.

6. The method of displaying heart rate variability of claim 5, further including the step of turning off the alarm if at least part of the heart rate variability is displayed above or is more than the minimum heart rate variability threshold after the heart rate variability is displayed below or is less than the minimum heart rate variability.

7. The method of displaying heart rate variability of claim 1, wherein heart rate variability is measured by one of beats per minute, frequency power, percentage of beat intervals over a period of time, entropy measure, peak frequency, and correlation dimension.

8. The method of displaying heart rate variability of claim 1, wherein the heart rate variability is displayed on at least one graph disposed on the display device as a magnitude from zero.

9. The method of displaying heart rate variability of claim 1, wherein the step of superimposing includes at least one color identified on the display device, the at least one color configured to indicate the heart rate variability level has decreased below a threshold value over a period of time.

10. The method of displaying heart rate variability of claim 1, further including the step of triggering an alarm indicating that the patient is at an increased risk of mortality or morbidity when the at least one color on the display device is superimposed and the heart rate variability level is maintained below the threshold value.

11. A system for displaying heart rate and heart rate variability, the system comprising:
a heart rate monitor configured to receive information indicative of at least a heart rate of a patient;
a processor circuit coupled to the heart rate monitor, the processor circuit configured to:
receive a first signal comprising a heart rate of a patient;
receive a second signal comprising a heart rate variability for the patient, wherein the heart rate variability is a standard deviation of the heart rate variability from a mean value over a period of time;
display the heart rate and the heart rate variability on a display device; and
wherein the display device includes at least one indicator corresponding to the heart rate variability superimposed on the display device such that a patient status corresponding to the at least one indicator is identified when the second signal is above or below a threshold value.

12. The system of claim 11, wherein the display device is configured to superimpose the at least one indicator on a colored background when the heart rate variability decreases over a period of time below the threshold value.

13. The system of claim 12, wherein the processor circuit is configured to activate an alarm when the heart rate variability decreases below the threshold value and is configured to deactivate the alarm when the heart rate variability increases above the threshold value.

14. The system of claim 11, wherein the display device is configured to display the heart rate variability on a bar chart, wherein a first color indicates a first level of the heart rate variability, and a second color indicates a second level of the heart rate variability, the second level being higher than the first level.

15. The system of claim 14, wherein the processor circuit is configured to activate an alarm when the first level of the heart rate variability represents the first color and is configured to deactivate the alarm when the second level of the heart rate variability represents the second color.

16. The system of claim 11, wherein a bar chart displaying the heart rate variability includes a plurality of colors, wherein each color of the plurality of colors is designated for a different level of heart rate variability risk corresponding to a patient status, wherein the plurality of colors includes a first bar color configured to display a first level of heart rate variability risk, a second bar color configured to display a second level of heart rate variability risk, and a third bar color configured to display a third level of heart rate variability risk, wherein each of the first bar color, the second bar color, and the third bar color represent different colors to quantify the patient status.

17. A method of displaying heart rate variability and managing heart rate variability alarms, the method comprising the following steps:
obtaining a heart rate value from a patient using information about patient heart beats over a time period;
obtaining a heart rate variability using a mean value of the heart rate value over the time period;
displaying the heart rate value and the heart rate variability on a display device;
identifying a minimum threshold value for the heart rate variability and marking the minimum threshold value on the display device;
superimposing at least one indicator corresponding to the minimum threshold value over the heart rate variability on the display device;
quantifying a patient status to a clinician, wherein the patient status corresponds to the at least one indicator superimposed over the heart rate variability; and
triggering an alarm into an on position when the heart rate variability is equivalent to or below the minimum threshold value and an off position when the heart rate variability exceeds the minimum threshold value.

18. The method of displaying heart rate variability and managing heart rate variability alarms of claim 17, wherein the heart rate value is displayed on a first graph of the display device and the heart rate variability is displayed on a second graph of the display device separate from the first graph such that the heart rate value and the heart rate variability are separately displayed on the display device.

19. The method of displaying heart rate variability and managing heart rate variability alarms of claim 17, wherein the heart rate value and the heart rate variability are displayed on a first graph of the display device such that the heart rate value and the heart rate variability are graphed together on the display device.

20. The method of displaying heart rate variability and managing heart rate variability alarms of claim 17, further including the step of superimposing at least one color, at least one line, or at least one region on the display device, and the heart rate variability is identified with one of the at least color, at least one line, or the at least one region based on the value of the heart rate variability when the heart rate variability is above or below the minimum threshold value.
